(19) United States
(12) Patent Application Publication
(10) Pub. No.: US 2008/0085505 A1
(43) Pub. Date: Apr. 10, 2008

(54) Method for stimulation of bioluminescent organisms via turbulence created by gas bubbles

(76) Inventors: Bryan Bjorndal, Carlsbad, CA (US); David Lapota, San Diego, CA (US)

Correspondence Address: OFFICE OF PATENT COUNSEL, SPAWARSYCEN, CODE 360012, 53510 SILVERGATE AVE. ROOM 103, SAN DIEGO, CA, 92152-5765, US

(21) Appl. No.: 11/586,747

(22) Filed: Oct. 10, 2006

(51) Int. Cl. C12Q 1/00; C12M 3/00

(52) U.S. Cl. 435/4; 435/287.1

(57) ABSTRACT

A method for stimulating bioluminescent organisms comprising the steps of forming gas bubbles in an aqueous suspension comprising the bioluminescent organisms; stimulating the bioluminescent organisms with the gas bubbles; and then measuring a characteristic of light emitted by the stimulated bioluminescent organisms.

### CROSS-REFERENCE TO RELATED APPLICATIONS

This application is related to U.S. application Ser. No. UNKNOWN, filed EVEN DATE, entitled “System and Method for Quantifying Toxicity in Water, Soil, and Sediments” (Navy Case #98125).

### FEDERALLY-SPONSORED RESEARCH AND DEVELOPMENT

This invention was developed with federal funds and is assigned to the United States Government. Licensing and technical inquiries may be directed to the Office of Patent Counsel, Space and Naval Warfare Systems Center, San Diego, Code 20012, San Diego, Calif., 92152; telephone (619) 553-3001, facsimile (619) 553-3821. Reference Navy Case No. 98122.

### BACKGROUND OF THE INVENTION

Bioluminescent organisms have the ability to produce a visible light when stimulated. Current methods of stimulating bioluminescent organisms typically involve either mechanically stirring an aqueous suspension containing the organisms or exposing the organisms to ultrasound. Over time, mechanically stirring an aqueous suspension leads to corrosion of shafts, propellers, and other moving parts in contact with the water. Although exposing the organisms to ultrasound solves some of the corrosion problems, ultrasound requires greater amounts of energy than stirring. An energy-efficient method is needed for stimulating bioluminescent organisms without exposing moving parts to water.

### BRIEF DESCRIPTION OF THE DRAWINGS

Throughout the several views, like elements are referenced using like references. Figures are not drawn to scale.

FIG. 1 is a flow chart of a method for stimulating bioluminescent organisms.

FIG. 2 illustrates an embodiment of a method for stimulating bioluminescent organisms.

FIG. 3 illustrates an embodiment of a method for stimulating bioluminescent organisms.

FIG. 4 illustrates an embodiment of a method for stimulating bioluminescent organisms.

FIG. 5 illustrates an embodiment of a method for stimulating bioluminescent organisms.

### DETAILED DESCRIPTION OF EMBODIMENTS

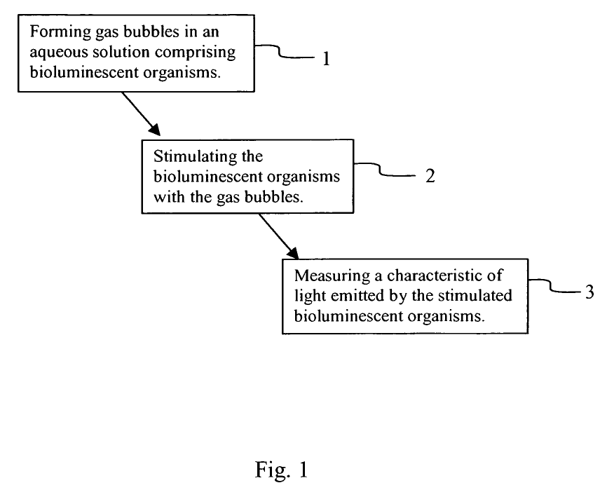

FIG. 1 is a flow chart embodying the steps of a method for stimulating bioluminescent organisms (BLOs) 10. Step 1 involves forming gas bubbles 20 in an aqueous suspension 30 comprising BLOs 10. Step 2 involves stimulating the BLOs 10 with the gas bubbles 20. Step 3 involves measuring a characteristic of light emitted by the stimulated BLOs 10.

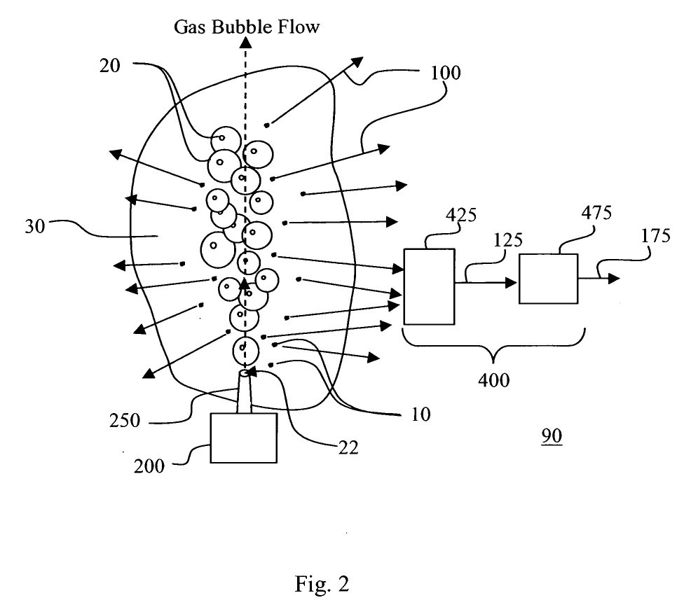

FIG. 2 shows gas bubbles 20, comprised of gas 22, formed in aqueous suspension 30. In one embodiment, the gas bubbles 20 may be formed in aqueous suspension 30 by injecting a gas 22 into aqueous suspension 30. In another embodiment, gas bubbles 20 may be formed in aqueous suspension 30 by inserting a material into aqueous suspension 30 that releases gas 22 upon contact with aqueous suspension 30. For example, solidified carbon dioxide may be inserted into aqueous suspension 30 to form gas bubbles 20 of carbon dioxide gas. The gas 22 may be provided by a gas source 200 and injected into aqueous suspension 30 via a gas deliverer 250. The gas bubbles 20 may be of any type of gas. By way of example, suitable gases for the gas bubbles 20 include, but are not limited to, air, nitrogen, oxygen, and carbon dioxide. The gas source 200 may be any source of gas capable of providing gas 22 that may be formed into gas bubbles 20 in aqueous suspension 30. Suitable embodiments for the gas source 200 include, but are not limited to, a compressed gas reservoir, and a gas pump. For example, in one embodiment, the gas source 200 may be a battery-powered air pump that receives air from the surrounding environment 90 and pumps the air bubbles 20 into aqueous suspension 30. The bubble deliverer 250 may be a tube, a nozzle, a pipe, or any other device capable of transporting gas 22 from the gas source 200 and injecting gas 22 into aqueous suspension 30 to form gas bubbles 20.

Forming gas bubbles 20 in aqueous suspension 30 creates turbulence, which induces fluid shear stress in aqueous suspension 30, which serves to stimulate the BLOs 10 to emit bioluminescent light 100. Fluid shear stress may be defined as a change in direction or pressure of the water surrounding the BLOs 10. In one embodiment, turbulence may be created when the ratio X/V ranges from about 0.667 to about 6.667, where X equals the flow rate of gas 22 into the aqueous suspension 30, and V equals the volume of aqueous suspension 30. For example, in one embodiment, the volume of aqueous suspension 30 may be 3 milliliters and the flow rate of gas bubbles 20 into aqueous suspension 30 may be 7 milliliters per second. Injecting gas 22 into aqueous suspension 30 such that gas 22 forms gas bubbles 20 may serve as a method of mixing BLOs 10 throughout aqueous suspension 30. In other words, the BLOs 10 may be distributed throughout aqueous suspension 30 by gas bubbles 20.

A characteristic of the bioluminescent light 100 emitted by the BLOs 10 due to stimulation by the gas bubbles 20 may be measured by a measuring unit 400. BLOs 10 may be any organisms that are capable of emitting bioluminescent light 100 in response to fluid shear stress in aqueous suspension 30. Dinoflagellates, such as Gonyaulax polyedra, Pyrocystis lunula, Pyrocystis fusiformis, and Pyrodinium bahamense are suitable examples of BLOs 10. BLOs 10 may be from marine environments.

Characteristics of bioluminescent light 100 that may be measured by measuring unit 400 include, but are not limited to, intensity, wavelength, photon count, and duration. As shown in FIG. 2, the measuring unit 400 may comprise a detector 425 and an analyzer 475. The detector 425 may be capable of transforming bioluminescent light 100 into signal 125. Signal 125 may then be transmitted to analyzer 475. The analyzer 475 may then quantify and/or measure a characteristic of signal 125 and produce output data 175. For example, if the photon count is the characteristic of bioluminescent light 100 that is to be measured, for every photon, or cluster of photons, detected by the detector 425 the signal 125 may be transmitted to the analyzer 475. The analyzer 475 may then quantify the number of signals 125 and generate output data 175 representative of the number of photons detected by detector 425. The output data 175 may be communicated to a user or serve as an input for another function within the analyzer 475. In another example, if the intensity of bioluminescent light 100 is the characteristic to be measured, the detector 425 may detect and transform bioluminescent light 100 into signal 125. Signal 125 may be transmitted to the analyzer 475 where the intensity of signal 125 may be measured against either a standard value for intensity or against intensities of prior signals 125 recorded by the analyzer 475. The analyzer 475 may then generate output data 175, representative of the intensity of the bioluminescent light 100. A suitable embodiment for the detector 425 may be a photomultiplier tube, a photodiode, a charge-coupled device (CCD), or any other device capable of detecting bioluminescent light 100 and generating an output signal. The analyzer 475 may be a computer, a processor, or any other device capable of quantifying and/or measuring a characteristic of signal 125.

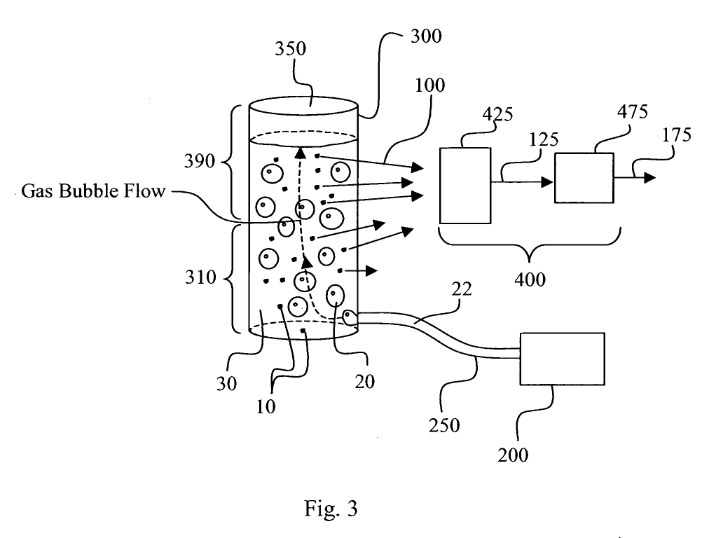

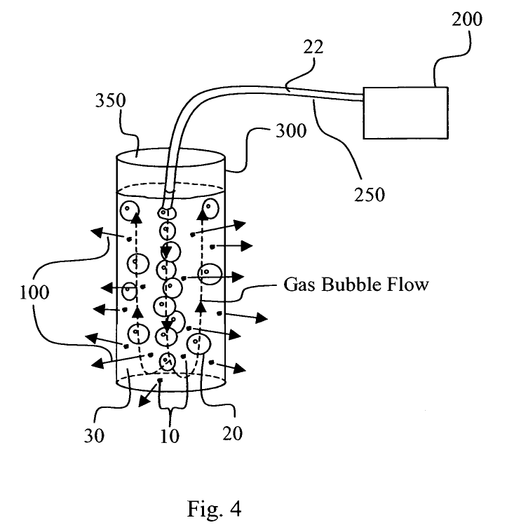

FIG. 3 shows aqueous suspension 30 contained in a container 300 having a lower end 310 and an upper end 390. The lower end 310 is shown in FIG. 3 as comprising about the lower 50% of the container 300. The upper end 390 is shown in FIG. 3 as about the upper 50% of the container 300. The container 300 may be any shape or size that allows for the measurement of a characteristic of bioluminescent light 100. For example, in one embodiment, the container 300 may be a 4.5-milliliter optical-grade, transparent, spectrophotometric cuvette. Other example embodiments include, but are not limited to, a graduated flask, a test tube, or a Petri dish. Gas 22 may be injected into suspension 30 from the upper end 390, as shown in FIG. 4, or the lower end 310, as shown in FIG. 3. In one embodiment, as depicted in FIG. 3, gas 22 may be injected into the lower end 310 thus creating turbulence in the suspension 30 as the gas bubbles 20 flow through suspension 30 towards the upper end 390 thus stimulating BLOs 10 to emit bioluminescent light 100. In another embodiment, as shown in FIG. 4, gas 22 may be injected into suspension 30 from the upper end 390 with sufficient force to allow the resulting gas bubbles 20 to penetrate into the lower end 310 before the gas bubbles 20 flow back up to the upper end 390. The gas 22 may exit the container 300 at the upper end 390 via a vent 350.

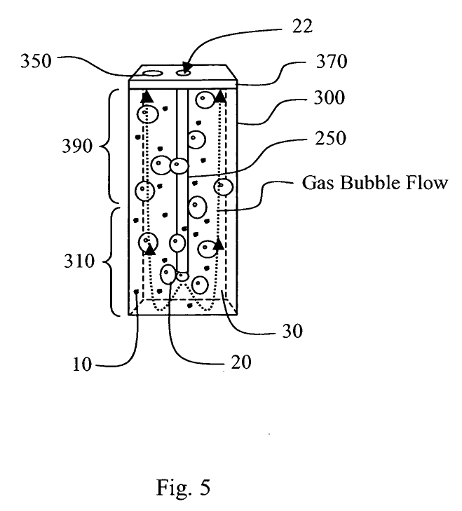

FIG. 5 shows the gas deliverer 250 extending down into the lower end 310 from the upper end 390 of container 300. The gas deliverer 250 is also shown as being connected to a cap 370. Cap 370 serves to prevent aqueous suspension 30 from escaping container 300, and also allows gas 22 to escape through vent 350. Gas 22 may be injected into aqueous suspension 30 via gas deliverer 250 according to the ratio X/V ranges from about 0.667 to about 6.667, where X equals the flow rate of gas 22 into the aqueous suspension 30, and V equals the volume of aqueous suspension 30. The gas source 200 and the measuring unit 400 have not been shown in FIG. 5 for the sake of clarity.

From the above description of the method for stimulation of bioluminescent organisms via turbulence created by gas bubbles, it is manifest that various techniques may be used for implementing the concepts of the method without departing from its scope. The described embodiments are to be considered in all respects as illustrative and not restrictive. It should also be understood that the method for stimulation of bioluminescent organisms via turbulence created by gas bubbles is not limited to the particular embodiments described herein, but is capable of many embodiments without departing from the scope of the claims.

We claim:

1. A method for stimulating bioluminescent organisms comprising:
forming gas bubbles in an aqueous suspension comprising said bioluminescent organisms;
stimulating said bioluminescent organisms with said gas bubbles; and
measuring a characteristic of light emitted by said stimulated bioluminescent organisms.

2. The method of claim 1, further comprising:
providing a gas; and
injecting said gas into said aqueous suspension to form said gas bubbles.

3. The method of claim 2, wherein:
X/V is in the range of about 0.667 to about 6.667, wherein X is an injection rate of said gas into said aqueous suspension, and V is the volume of said aqueous suspension.

4. The method of claim 1, wherein the intensity of said light is measured.

5. The method of claim 1, wherein the polarization state of said light is measured.

6. The method of claim 1, wherein the wavelength of said light is measured.

7. The method of claim 1, wherein the duration of said light is measured.

8. The method of claim 1, wherein said bioluminescent organisms are dinoflagellates.

9. The method of claim 1, wherein said bioluminescent organisms are Gonyaulax polyedra.

10. The method of claim 1, wherein said bioluminescent organisms are Pyrocystis lunula.

11. The method of claim 1, wherein said gas is supplied by a battery powered air pump.

12. The method of claim 1, wherein said measuring step is performed with a measuring unit.

13. The method of claim 12, wherein said measuring unit comprises a detector and an analyzer.

14. The method of claim 13, wherein said detector is a photodiode.

15. The method of claim 14, wherein said analyzer is a computer.

16. The method of claim 3, wherein said aqueous suspension is contained in a container having a lower and an upper end, and said gas is injected into said aqueous suspension from said lower end.

17. The method of claim 16, wherein said container is a transparent cuvette.

18. The method of claim 16, further comprising:
allowing said gas to escape through a vent in said upper end.

19. A method for mixing comprising:
providing a gas;
injecting said gas into an aqueous suspension comprising bioluminescent organisms, wherein said gas forms gas bubbles in said aqueous suspension;
mixing said bioluminescent organisms throughout said aqueous suspension with said gas bubbles.

20. The method of claim 19, wherein:
X/V is in the range of about 0.667 to about 6.667, wherein X is an injection rate of said gas into said aqueous suspension, and V is the volume of said aqueous suspension.
